(19) United States
(12) Patent Application Publication
(10) Pub. No.: US 2007/0231788 A1
(43) Pub. Date: Oct. 4, 2007

(54) Method for In Vitro Assay of Demineralized Bone Matrix

(76) Inventors: Keyvan Behnam, Red Bank, NJ (US); Haiping Tang, Eatontown, NJ (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/683,938

(22) Filed: Mar. 8, 2007

Related U.S. Application Data
(63) Continuation-in-part of application No. 10/584,981, filed on Jun. 29, 2006.
(60) Provisional application No. 60/533,537, filed on Dec. 31, 2003.

(51) Int. Cl. G01N 33/00

(52) U.S. Cl. 435/4

(57) ABSTRACT

A sensitive, quantitative method to assess bone forming potential of demineralized bone matrix (DBM) is provided. Cortical DBM is treated with collagenase. The treated cortical DBM is placed in a cell culture for a dwell time. After the dwell time, the cell culture is observed for osteoblastic markers. The cortical bone so treated exhibits the same bone forming potential in vivo as untreated cortical bone.

This application is a continuation-in-part of U.S. patent application Ser. No. 10/584,981, filed Jun. 29, 2006, which is a 35 U.S.C. § 371 filing of International Application No. PCT Application No. PCT/US2004/043999, filed Dec. 31, 2004, which in turn claims priority to U.S. Provisional Application No. 60/533,537, filed Dec. 31, 2003, each of which is hereby incorporated by reference.

### BACKGROUND

### Introduction

Mammalian bone tissue is known to contain one or more proteinaceous materials, presumably active during growth and natural bone healing, that can induce a developmental cascade of cellular events resulting in endochondral bone formation. The active factors variously have been referred to in the literature as bone morphogenetic or morphogenic proteins (BMPs), bone inductive proteins, bone growth or growth factors, osteogenic proteins, or osteoinductive proteins. These active factors are collectively referred to herein as osteoinductive factors.

It is well known that bone contains these osteoinductive factors. These osteoinductive factors are present within the compound structure of cortical bone and are present at very low concentrations, e.g., 0.003%. Osteoinductive factors direct the differentiation of pluripotential mesenchymal cells into osteoprogenitor cells that form osteoblasts. Based upon the work of Marshall Urist as shown in U.S. Pat. No. 4,294,753, issued Oct. 13, 1981, proper demineralization of cortical bone exposes the osteoinductive factors, rendering it osteoinductive, as discussed more fully below.

The ability of demineralized bone matrix (DBM) to induce bone formation varies depending on the treatment of the DBM. A variety of methods have been developed to assess the bone forming potential of the DBM.

### Overview of Bone Grafts

The rapid and effective repair of bone defects caused by injury, disease, wounds, or surgery has long been a goal of orthopaedic surgery. Toward this end, a number of compositions and materials have been used or proposed for use in the repair of bone defects. The biological, physical, and mechanical properties of the compositions and materials are among the major factors influencing their suitability and performance in various orthopaedic applications.

Autologous cancellous bone (“ACB”) long has been considered the gold standard for bone grafts. ACB may be osteoinductive and nonimmunogenic, and should have all of the appropriate structural and finctional characteristics appropriate for the particular recipient. Unfortunately, ACB is only available in a limited number of circumstances. Some individuals lack ACB of appropriate dimensions and quality for transplantation, and donor site pain and morbidity can pose serious problems for patients and their physicians.

Much effort has been invested in the identification and development of alternative bone graft materials. Urist has published seminal articles on the theory of bone induction and a method for decalcifying bone, i.e., making DBM. Urist M. R., Bone Formation by Autoinduction, Science 1965, 150(698):893-9; Urist M. R. et al., The Bone Induction Principle, Clin. Orthop. Rel. Res. 53:243-283, 1967. As mentioned above, it is known that DBM that is derived from cortical bone is an osteoinductive material, in that it induces bone growth when implanted in an ectopic site of a rodent, owing to the osteoinductive factors contained within the DBM. Honsawek et al. (2000). It is also known that there are numerous osteoinductive factors, including some of BMP 1-15, which are part of the transforming growth factor-beta (TGF-beta) superfamily. Kawabata et al., 2000. BMP-2 has become the most important and widely studied of the BMP family of proteins. There are also other proteins present in DBM that are not osteoinductive alone but still contribute to bone growth, including fibroblast growth factor-2 (FGF-2), insulin-like growth factor-I and -II (IGF-I and IGF-II), platelet derived growth factor (PDGF), and transforming growth factor-beta 1 (TGF-beta.1). Hauschka, et al. 1986; Canalis, et al, 1988; Mohan et al. 1996.

DBM implants have been reported to be particularly useful (see, for example, U.S. Pat. Nos. 4,394,370, 4,440,750, 4,485,097, 4,678,470, and 4,743,259; Mulliken et al., Calcif. Tissue Int. 33:71, 1981; Neigel et al., Opthal. Plast. Reconstr. Surg. 12:108, 1996; Whiteman et al., J. Hand. Surg. 18B:487, 1993; Xiaobo et al., Clin. Orthop. 293:360, 1993, each of which is incorporated herein by reference). DBM typically is derived from cadavers. The bone is removed aseptically and treated to kill any infectious agents. The bone is particulated by milling or grinding, and then the mineral component is extracted by various methods, such as by soaking the bone in an acidic solution. The remaining matrix is malleable and can be further processed and/or formed and shaped for implantation into a particular site in the recipient. Demineralized bone prepared in this manner contains a variety of components including proteins, glycoproteins, growth factors, and proteoglycans. Following implantation, the presence of DBM induces cellular recruitment to the site of injury. The recruited cells may eventually differentiate into bone forming cells. Such recruitment of cells leads to an increase in the rate of wound healing and, therefore, to faster recovery for the patient.

One study, looking at cortical bone matrix from monkeys, determined that monkey bone matrix induces ectopic bone formation in the athymic rat but not in adult monkeys. It was concluded that adult monkey bone matrix contains bone inductive properties but that these properties are not sufficient to induce bone formation in adult monkey muscle sites. Aspenberg et al., J. of Orthop. Res. 9:20-25, 1991.

Another study evaluated bone and cementum regeneration following guided tissue regeneration (GTR) in periodontal fenestration defects. Specifically, the adjunctive effect of allogenic, freeze-dried DBM implants was evaluated and found to exhibit no discernible adjunctive effect to GTR in the defect model. The critical findings were 1) the DBM particles remained embedded in dense connective tissue without evidence of bone metabolic activity; and 2) limited and similar amounts of bone and cementum regeneration were observed for both GTR plus DBM and GTR defects. Caplanis et al., J Periodontal 851-856, August, 1998.

Various papers have looked at cartilage tissue differentiation of bone matrix gelatin (BMG) from cortical bone. Terashima and Urist found that cortical bone BMG is chemically more reactive than whole bone matrix. Terashima et al., Chondrogenesis in Outgrowths of Muscle Tissue onto Modified Bone Matrix in Tissue Culture, Clinical Orthopaedics and Related Research, No. 127, September 1977:248-256. A later study found that rat BMG can induce chondrogenesis in cell culture. Urist et al., Cartilage Tissue Differentiation from Mesenchymal Cells Derived from Mature Muscle in Tissue Culture, In Vitro, Vol. 14, No. 8, 1978:697-706. Nogami and Urist also assessed the effect of various treatments of cortical bone, including collagenase treatment of cortical bone BMG, on cartilage tissue differentiation. Nogami and Urist, Substrata Prepared from Bone Matrix for Chondrogenesis in Tissue Culture, The Journal of Cell Biology, Vol. 62, 1974:510-519.

### Bone Forming Potential Assays

Bone forming potential of DBM can be assessed in vivo or in vitro.

In Vivo Assays

In vivo, the amount of bone, the quality of bone, and the speed of formation are examined. Generally, the amount of cortical bone formation is a key indicator of bone forming potential. In vivo assessment may be done by implanting the DBM ectopically or orthotopically. Orthotopically, the DBM is implanted in a bone healing capacity. Ectopically, the bone is implanted at a site where bone would not generally be present. Typically this is done in an athymic rat or mouse.

Induction of bone formation can be determined by a histological evaluation showing the de novo formation of bone with accompanying osteoblasts, osteoclasts, and osteoid matrix. For example, osteoinductive activity of an osteoinductive factor can be demonstrated by a test using a substrate onto which material to be tested is deposited. The substrate with deposited material is implanted subcutaneously in a test animal. The implant is subsequently removed and examined microscopically for the presence of bone formation including the presence of osteoblasts, osteoclasts, and osteoid matrix. A suitable procedure for assessing osteoinductive activity is illustrated in Example 5 of U.S. Pat. No. 5,290,763, herein incorporated by reference. Although there is no generally accepted scale of evaluating the degree of osteogenic activity, certain factors are widely recognized as indicating bone formation. Such factors are referenced in the scale of 0-8, which is provided in Table 3 of Example 1 of U.S. Pat. No. 5,563,124, herein incorporated by reference. The 0-4 portion of this scale corresponds to the scoring system described in U.S. Pat. No. 5,290,763, which is limited to scores of 0-4. The remaining portion of the scale, scores 5-8, references additional levels of maturation of bone formation. The expanded scale also includes consideration of resorption of collagen, a factor which is not described in the '763 patent.

In studies, a typical amount of DBM for implantation may be 20 mg in a mouse and 40 mg in a rat. Significant increases in the growth factor dose, for example, 150× dose (or 150 times the growth factor found in normal DBM), lead to significantly more and potentially faster bone growth with larger volume bone growth, more dense bone growth, larger nodules of bone growth, higher x-ray density, and, generally, a higher osteoinductive score. Associated with this increase in osteoinductivity can be a cortical shell surrounding the nodule and some level of vascularization in the nodule. However, the ability to quantitatively measure is generally limited by the method used, and generally measured increases in osteoinductive activity are not linear with the increase in dosage. Thus, if 20 mg of DBM gives an osteoinductive activity of 1 on a scale of 1 to 100, 100 times the growth factor dose (2000 mg of DBM growth factors) does not give an osteoinductive activity of 100. Instead, it may result in an osteoinductive activity of about 20. Further, in addition to, or in lieu of, testing at 28 days, it may be desirable to test inductivity at 21 days. Generally, inductivity may be measured histomorphometrically by methods known in art.

The amount of bone growth generally saturates around 28 days. New bone may form quickly but quality is determined later. Typically newly formed bone is woven, having structural similarities to cancellous bone. Cortical bone formation is an indicator of high quality bone formation; cortical shelling is an extreme case of high quality bone formation. High osteoinductive activity generally results in a rim of cortical bone surrounding the new bone formation. Osteoid deposition next to the DBM particles in the culture is typical but, in cases of high activity, there may be contiguous bone formation or one large ossicle.

An osteoinductivity score is assigned pursuant to the in vivo assay. Generally, the osteoinductivity score is a score on a scale of 0 to 4 as determined according to the method of Edwards et al., “Osteoinduction of Human Demineralized Bone: Characterization in a Rat Model,” Clinical Orthopaedics & Rel. Res., 357:219-228, December 1998, incorporated herein by reference. In the method of Edwards et al, a score of “0” represents no new bone formation; “1” represents 1%-25% of implant involved in new bone formation; “2” represents 26-50% of implant involved in new bone formation; “3” represents 51%-75% of implant involved in new bone formation; and “4” represents >75% of implant involved in new bone formation. In most instances, the score is assessed 28 days after implantation. However, the osteoinductivity score may be obtained at earlier time points such as 7, 14, or 21 days following implantation. In these instances it may be desirable to include a normal DBM control such as DBM powder without a carrier, and if possible, a positive control such as BMP. Occasionally, osteoinductivity also may be scored at later timepoints such as 40, 60, or even 100 days following implantation.

A limitation of measurement using osteoinductive scores is that, in some situations, the system's ability to respond may be saturated. Thus, for example, if the score ranges only from 1 to 4, two samples may have the same score (4) but may not, in fact, be comparable. This is particularly the case when the bone resulting from one method or implant is qualitatively better than the bone resulting from another method or implant. That is, both methods or implants may result in an osteoinductive score of 4 but one may result in qualitatively better bone than the other. Thus, in some situations it may be desirable to test speed of growth, density, presence of cortical bone, shelling, and/or other factors showing an increase over normal demineralized bone matrix.

In vitro assays are less expensive than in vivo assays, and they may take less time. Prior art in vitro assays, however, have not been sensitive, and they have not provided reliable or reproducible results.

In vitro assays look at induction of osteogenic phenotypic markers or surrogates (such as cartilage). In vitro assays are generally cell culture assays wherein the DBM is added to a cell culture. The cell culture may be from a primary cell line, such as fat, bone, or muscle, of progenitor cells or stem cells. The cell culture may be an established cell line such as a clonal line, a myoblastic cell line such as C2C12, or C3HT10(1/2). A myoblastic cell line, such as C2C12, is a cell line that gives rise to muscle, particularly myotubes—large nucleated cells that result from fusion of mononucleated cells or immature muscle cells.

A period of time after addition of the DBM to the cell line, the regulation of osteoblastic markers in myoblastic cell lines is measured quantitatively as a function of specific enzyme activity or changes in protein or mRNA levels. The DBM may be in the cell line for days to weeks before measurement. In the process of bone formation, cells express additional osteoblastic marker that are associated with osteogenesis. For example, cells induce formation of alkaline phosphatase, an enzymatic osteoblastic marker. Measuring the presence of alkaline phosphatase gives an indication of the bone forming potential of the DBM. Various methods, including polymerase chain reaction, may be used for measuring osteoblastic markers.

Using cell culture methods known in the art, an assay typically takes approximately 14 days. The results of these methods are quantitative but are generally not normalized to an extent where a quantitative activity may be compared to another activity recorded in a different setting at a different time. Nevertheless, in order to illustrate the lack of sensitivity of the conventional assays on a hypothetical scale ranging from 1 to 100, DBM would typically exhibit activity having a score of only 1 to 4. A score of 1 is associated with inactive materials, such as DBM extracted with guanidine hydrochloride, where the growth factors have been removed. A score of 100 is associated with recombinant BMP at half strength (ED₅₀). Thus, it is difficult to assess variations in bone forming potential of DBM where the assay score will vary only subtly. Further, because of the very small range in which DBM typically falls, the tests are not typically reproducible. That is, testing of the DBM may give a score of 1 in one test and a score of 2 in another, thus providing little information as to the osteoinductive potential of the bone.

It would be useful to assay the bone forming potential of DBM in vitro in a sensitive, quantitative, and reproducible manner.

### BRIEF SUMMARY

A sensitive, quantitative method to assess the bone forming potential of DBM is provided. Cortical DBM is treated with collagenase prior to cell culture assay. The treated cortical bone exhibits the same bone forming potential in vivo as untreated cortical bone. In a cell culture assay, the treated cortical bone typically scores between approximately 50 and 100 on a scale ranging from 1 to 100.

In one embodiment, the method includes partially digesting demineralized cortical bone matrix with collagenase. The partially demineralized cortical bone matrix is placed in a cell culture and left in the culture for a dwell time. After the dwell time, the cell culture is observed for osteoblastic markers.

This application refers to various patents, patent applications, journal articles, and other publications, all of which are incorporated herein by reference. The following documents are incorporated herein by reference: PCT/US04/43999; PCT/US05/003092; US 2003/0143258 Al; PCT/US02/32941; Current Protocols in Molecular Biology, Current Protocols in Immunology, Current Protocols in Protein Science, and Current Protocols in Cell Biology, John Wiley & Sons, N.Y., edition as of July 2002; Sambrook, Russell, and Sambrook, Molecular Cloning: A Laboratory Manual, 3rd ed., Cold Spring Harbor Laboratory Press, Cold Spring Harbor, 2001; Rodd 1989 “Chemistry of Carbon Compounds,” Vols. 1-5 and Supps., Elsevier Science Publishers, 1989; “Organic Reactions,” Vols. 1-40, John Wiley and Sons, New York, N.Y., 1991; March 2001, “Advanced Organic Chemistry,” 5th ed. John Wiley and Sons, New York, N.Y. In the event of a conflict between the specification and any of the incorporated references, the specification shall control. Where numerical values herein are expressed as a range, endpoints are included.

While multiple embodiments are disclosed, still other embodiments of the present invention will become apparent to those skilled in the art from the following detailed description. As will be apparent, the method disclosed herein is capable of modifications in various obvious aspects, all without departing from the spirit and scope of the present teachings. Accordingly, the detailed description is to be regarded as illustrative in nature and not restrictive.

### BRIEF DESCRIPTION OF THE DRAWINGS

FIG. 1 illustrates a flow chart of a method for in vitro assay of the bone forming potential of demineralized cortical bone matrix.

FIG. 2 is a graph showing alkaline phosphate activity in C2C12 cells cultured with inactive DBM and alkaline phosphate activity in C2C12 cells cultured with active DBM using an embodiment of the method taught herein.

FIG. 3 is a graph showing the effects of collagenase digestion on specific alkaline phosphatase activity in C2C12 cells.

FIG. 4 is a graph showing total protein count after DBM or BMP treatment.

FIG. 5 illustrates alkaline phosphatase induction by DBM of different species.

FIG. 6 is a bar graph showing alkaline phosphate activity in C2C12 cells cultured with DBM using a method corresponding to the work of Peel et al., referenced below. Cells were treated with DBM using transwell inserts in α-MEM containing either 5% (left bars in each group) or 15% fetal bovine serum (FBS) (right bars in each group).

FIG. 7 is a bar graph showing specific alkaline phosphatase activity of C2C12 cells treated with various preparations of human DBM, FBS, or BMP-2 for 6 days. The various groups are as follows: Cont: culture media only; DBM: 100 mg of human DBM; Col iaDBM: 100 mg Collagenase-treated GuHCl inactivated human DBM; Col Cont: 100 mg DBM incubated in digestion buffer lacking collagenase and undergoing washing and neutralization steps; Col DBM: 100 mg Collagenase treated human DBM, BMP: 100 ng BMP-2 (refreshed at each feeding). Cells were grown in DMEM supplemented with 10% FBS, 0.284 mM ascorbate 2-phosphate and 10 mM betaglycerol phosphate.

FIG. 8 is a bar graph showing the effect of ascorbate 2-phosphate (Ascb) and beta-glycerol phosphate (BGP) on the in vitro activity of collagenase-treated DBM. Treatment groups are labeled as in FIG. 7. Each group was cultured either in DMEM containing 10% FBS (leftmost bars in each set of 3), or DMEM containing 10% FBS supplemented with 0.284 mM ascorbate 2-phosphate (middle bars in each set of 3), or DMEM containing 10% FBS supplemented with 0.284 mM ascorbate 2-phosphate and 10 mM beta-glycerol phosphate (rightmost bars in each set of 3).

FIG. 9 shows phase contrast photomicrographs of C2C12 cells treated with a) 10% FBS (untreated control), b) 100 mg collagenase treated inactivated human DBM, and c) 100 mg collagenase-treated human DBM. All cells were cultured for 6 days in DMEM containing 10% FBS. Collagenase-treated human DBM and collagenase-treated inactivated DBM were added to 24-well plates using 8 μm transwell inserts.

FIG. 10 is a bar graph that shows DBM residue recovered from cell culture inserts after 6 days of tissue culture.

FIG. 11 shows Toluidine Blue-stained histology sections of heterotopic nodules that resulted following implantation of human BMG (A) or human DBM (B) into rat muscle. Forty mg of human DBM or 40 mg of human BMG was implanted in the quadriceps muscle of 6 week old female Harlan athymic rats (rnu/rnu). Twenty-eight days after surgery the nodules were recovered, and histological sections were prepared and stained with Toluidine Blue to allow visualization of residual bone matrix (indicated by arrows), along with new osteoid, bone marrow, and cartilage.

FIG. 12 illustrates a correlation of in vitro alkaline phosphatase activity with in vivo osteoinductivity.

### DEFINITIONS

Biocompatible, as used herein, is intended to describe materials that, upon administration in vivo, do not induce undesirable long-term effects.

Bone as used herein refers to bone that is cortical, cancellous or cortico-cancellous of autogenous, allogenic, xenogenic, or transgenic origin.

Demineralized bone, as used herein, refers to material generated by removing mineral material from bone tissue. The DBM compositions as used herein may include preparations containing less than 5% calcium, or less than 1% calcium by weight. Partially demineralized bone (e.g., preparations with greater than 5% calcium by weight but containing less than 100% of the original starting amount of calcium) are also considered within the scope of the present teachings. Superficially demineralized refers to bone-derived elements possessing at least about 90 weight percent of their original inorganic mineral content. Partially demineralized refers to bone-derived elements possessing from about 8 to about 90 weight percent of their original inorganic mineral content. Fully demineralized refers to bone containing less than 8% of its original mineral context. Demineralized bone encompasses such expressions as “substantially demineralized,” “partially demineralized,” “superficially demineralized,” and “fully demineralized.”

Marker, as used herein, refers to a molecular moiety (e.g., protein, peptide, MRNA or other RNA species, DNA, lipid, carbohydrate) that characterizes, indicates, or identifies one or more cell type(s), tissue type(s), or embryological origin. A cellular marker may, but need not be, cell type specific. For example, a cell type specific marker is generally a protein, peptide, MRNA, lipid, or carbohydrate that is present at a higher level on or in a particular cell type or cell types of interest than on or in many other cell types. In some instances a cell type-specific marker is present at detectable levels only on or in a particular cell type of interest. However, it will be appreciated that useful markers need not be absolutely specific for the cell type of interest. In general, a cell type-specific marker for a particular cell type is expressed at levels at least three-fold greater in that cell type than in a reference population of cells, which may consist, for example, of a mixture containing cells from a plurality (e.g., 5-10 or more) of different tissues or organs in approximately equal amounts. The cell type specific marker also may be present at levels at least 4-5 fold, between 5-10 fold, or more than 10-fold greater than its average expression in a reference population. Detection or measurement of a cell type specific marker may make it possible to distinguish the cell type or types of interest from cells of many, most, or all other types. In general, the presence and/or abundance of most markers may be determined using standard techniques such as Northern blotting, in situ hybridization, RT-PCR, sequencing, microarray analysis, immunological methods such as immunoblotting, immunodetection, or fluorescence detection following staining with fluorescently labeled antibodies, oligonucleotide or cDNA microarray or membrane array, protein microarray analysis, mass spectrometry, etc. Markers of interest include markers characteristic of bone and/or cartilage-forming cells. Alkaline phosphatase is one such marker.

Osteoconductive is used herein to refer to the ability of a non-osteoinductive substance to serve as a suitable template or substance along which bone may grow.

Osteogenic is used herein to refer to the ability of an agent, material, or implant to enhance or accelerate the growth of new bone tissue by one or more mechanisms such as osteogenesis, osteoconduction, and/or osteoinduction.

Osteoinductive, as used herein, refers to the quality of being able to recruit cells from the host that have the potential to stimulate new bone formation. Any material that can induce the formation of ectopic bone in the soft tissue of an animal is considered osteoinductive. For example, most osteoinductive materials induce bone formation in athymic rats when assayed according to the method of Edwards et al., 1998. In other instances, osteoinduction is considered to occur through cellular recruitment and induction of the recruited cells to an osteogenic phenotype.

Proteases, as used herein, are protein-cleaving enzymes that cleave peptide bonds that link amino acids in protein molecules to generate peptides and protein fragments. A large collection of proteases and protease families has been identified. Some exemplary proteases include serine proteases, aspartyl proteases, acid proteases, alkaline proteases, metalloproteases, carboxypeptidase, aminopeptidase, cysteine protease, collagenase, etc. An exemplary family of proteases is the proprotein convertase family, which includes furin. Dubois et al., American Journal of Pathology (2001) 158(1):305316. Members of the proprotein convertase family of proteases are known to proteolytically process proTGFs and proBMPs to their active mature forms. Dubois et al., American Journal of Pathology (2001) 158(1):305-316; Cui et al., The Embo Journal (1998) 17(16):4735-4743; Cui et al, Genes & Development (2001) 15:2797-2802, each incorporated by reference herein.

Protease inhibitors, as used herein, refers to chemical compounds capable of inhibiting the enzymatic activity of protein cleaving enzymes (i.e., proteases). The proteases inhibited by these compounds include serine proteases, acid proteases, metalloproteases, carboxypeptidase, aminopeptidase, cysteine protease, etc. The protease inhibitor may act specifically to inhibit only a specific protease or class of proteases, or it may act more generally by inhibiting most if not all proteases. Suitable protease inhibitors are protein- or peptide-based and are commercially available from chemical companies such as Aldrich-Sigma. Protein or peptide-based inhibitors adhere to the DBM (or calcium phosphate or ceramic carrier), which remain associated with the matrix providing a stabilizing effect for a longer period of time than freely diffusible inhibitors, also may be used. Examples of protease inhibitors include aprotinin, 4-(2aminoethyl) benzenesulfonyl fluoride (AEBSF), amastatin-HC1, alphal-antichymotrypsin, antithrombin III, alphal-antitrypsin, 4-aminophenylmethane sulfonyl-fluoride (APMSF), arphamenine A, arphamenine B, E-64, bestatin, CA-074, CA-074-Me, calpain inhibitor I, calpain inhibitor II, cathepsin inhibitor, chymostatin, diisopropylfluorophosphate (DFP), dipeptidylpeptidase IV inhibitor, diprotin A, E-64c, E-64d, E-64, ebelactone A, ebelactone B, EGTA, elastatinal, foroxymithine, hirudin, leuhistin, leupeptin, alpha2macroglobulin, phenylmethylsulfonyl fluo4de (PMSF), pepstatin A, phebestin, 1,10phenanthroline, phosphoramidon, chymostatin, benzamidine HCI, antipain, epsilon aminocaproic acid, N-ethylmaleimide, trypsin inhibitor, 1-chloro-3-tosylamido-7-amino2-heptanone (TLCK), 1-chloro-3-tosylamido-4-phenyl-2-butanone (TPCK), trypsin inhibitor, and sodium EDTA.

Stabilizing agent is any chemical entity that, when included in an inventive composition comprising DBM and/or a growth factor, enhances the osteoinductivity of the composition as measured against a specified reference sample. In most cases, the reference sample will not contain the stabilizing agent, but in all other respects will be the same as the composition with stabilizing agent. The stabilizing agent also generally has little or no osteoinductivity of its own and works either by increasing the half-life of one or more of the active entities within the inventive composition as compared with an otherwise identical composition lacking the stabilizing agent, or by prolonging or delaying the release of an active factor. In certain embodiments, the stabilizing agent may act by providing a barrier between proteases and sugar-degrading enzymes thereby protecting the osteoinductive factors found in or on the matrix from degradation and/or release. In other embodiments, the stabilizing agent may be a chemical compound that inhibits the activity of proteases or sugar-degrading enzymes. In one embodiment, the stabilizing agent retards the access of enzymes known to release and solubilize the active factors. Half-life may be determined by immunolgical or enzymatic assay of a specific factor, either as attached to the matrix or extracted there from. Alternatively, measurement of an increase in osteoinductivity half-life, or measurement of the enhanced appearance of products of the osteoinductive process (e.g., bone, cartilage or osteogenic cells, products or indicators thereof) is a useful indicator of stabilizing effects for an enhanced osteoinductive matrix composition. The measurement of prolonged or delayed appearance of a strong osteoinductive response will generally be indicative of an increase in stability of a factor coupled with a delayed unmasking of the factor activity.

### DETAILED DESCRIPTION

I. Introduction

A sensitive, quantitative method to assess the bone forming potential of DBM is provided. Cortical DBM is treated with collagenase prior to cell culture assay. The treated cortical bone generally exhibits the same bone forming potential in vivo as untreated cortical bone. In a cell culture assay, the treated cortical bone typically scores between approximately 50 and 100 on a scale ranging from 1 to 100. Those of ordinary skill will appreciate that a variety of embodiments or versions of methods to assess the bone forming potential DBM in accordance with teachings herein are not specifically discussed below but are nonetheless within the scope of the disclosure, as defined by the appended claims.

Bone is made up cells, and also of collagen, minerals, and other noncollagenous proteins. Cortical bone, which accounts for approximately eighty percent of skeletal bone mass, is found in the hard outer layer of bone. Cortical bone is structural and bears the majority of the body's weight. Cancellous bone is the porous and spongy inner structure accounting for approximately twenty percent of skeletal bone mass. Cancellous bone contains bone marrow and the elements that bone uses to heal itself.

II. Overview

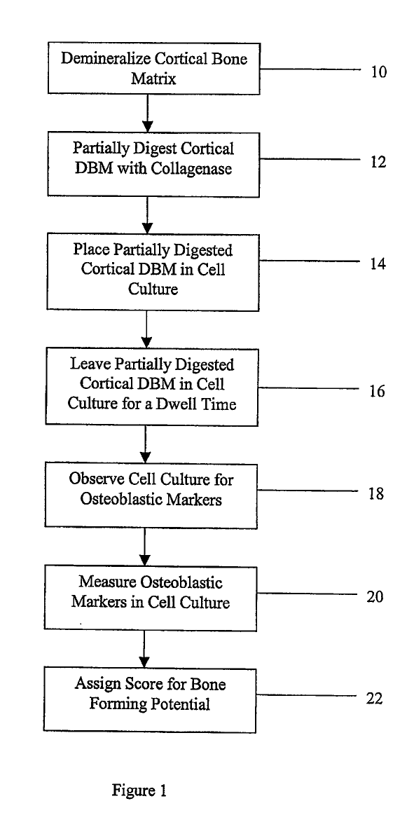

FIG. 1 illustrates a flow chart of a method for assaying the bone forming potential of cortical bone matrix. The cortical bone matrix is demineralized, shown at block 10. The demineralized bone matrix comprises cortical bone demineralized in any suitable manner. The DBM is partially digested with collagenase, shown at block 12. After treatment with collagenase, the DBM is used in a cell culture assay, such as via addition to a C2C12 cell line. Thus, the partially digested cortical bone matrix is placed in cell culture, shown at block 14, and left in the culture for a dwell time, shown at block 16. After the time has elapsed, the cell culture is observed for osteoblastic markers, shown at block 18. The osteoblastic markers are assessed and measured, shown at block 20, and a score, ranging from 0 to 100, is assigned, shown at block 22.

III. Providing Demineralized Bone

a. Demineralizing the Bone

The cortical bone matrix is demineralized, shown at block 10 of FIG. 1. The demineralized bone matrix may be provided in any suitable manner. The bone useful in the method disclosed herein may be obtained utilizing methods well known in the art, e.g., allogenic donor bone. Bone-derived elements can be readily obtained from donor bone by various suitable methods, e.g., as described in U.S. Pat. No. 6,616,698, incorporated herein by reference. The bone may be of autogenous, allogenic, xenogenic, or transgenic origin. Where the bone is from a nonhuman animal source, any suitable source for the application may be used, including bovine, ovine, canine, rabbit, equine, etc.

DBM preparations have been used for many years in orthopaedic medicine to promote the formation of bone. Typically, DBM preparations for promoting the formation of bone have comprised cortical or corticocancellous DBM. DBM has found use, for example, in the repair of fractures, in the fusion of vertebrae, in joint replacement surgery, and in treating bone destruction due to underlying disease such as rheumatoid arthritis. Cortical DBM is thought to promote bone formation in vivo by osteoconductive and osteoinductive processes. The osteoinductive effect of implanted cortical DBM compositions is thought to result from the presence of active growth factors present on the isolated collagen-based matrix. These factors include members of the TGF-β, IGF, and BMP protein families. Particular examples of osteoinductive factors include TGF-β, IGF-1, IGF-2, BMP-2, BMP-7, parathyroid hormone (PTH), and angiogenic factors. Other osteoinductive factors such as osteocalcin and osteopontin are also likely to be present in DBM preparations as well. There also are likely to be other unnamed or undiscovered osteoinductive factors present in DBM.

In a demineralization procedure in accordance with one embodiment, the bone is subjected to an acid demineralization step and a defatting/disinfecting step. The bone is immersed in acid over time to effect demineralization. Acids that can be employed in this step include inorganic acids such as hydrochloric acid and as well as organic acids such as formic acid, acetic acid, peracetic acid, citric acid, propionic acid, etc. The depth of demineralization into the bone surface can be controlled by adjusting the treatment time, temperature of the demineralizing solution, concentration of the demineralizing solution, and agitation intensity during treatment.

The demineralized bone is rinsed with sterile water and/or buffered solution(s) to remove residual amounts of acid and thereby raise the pH. A suitable defatting/disinfectant solution is an aqueous solution of ethanol, the ethanol being a good solvent for lipids and the water being a good hydrophilic carrier to enable the solution to penetrate more deeply into the bone particles. The aqueous ethanol solution also disinfects the bone by killing vegetative microorganisms and viruses. Ordinarily, at least about 10 to 40 percent by weight of water (i.e., about 60 to 90 weight percent of defatting agent such as alcohol) may be present in the defatting disinfecting solution to produce enhanced lipid removal and disinfection within the shortest period of time. A suitable concentration range of the defatting solution is from about 60 to about 85 weight percent alcohol, or about 70 weight percent alcohol. In one embodiment, the cancellous bone is defatted in a solution of 1:1 chloroform:methanol at room temperature and then demineralized in 0.6 N HCl at 4° C.

The DBM may be ground or otherwise processed into particles of an appropriate size before or after demineralization. In certain embodiments, the particle size is greater than 75 microns, ranges from about 100 to about 3000 microns, or ranges from about 100 to about 800 microns. After grinding the DBM to the desired size, if done, the mixture may be sieved to select those particles of a desired size. In certain embodiments, the DBM particles may be sieved though a 50 micron sieve, a 75 micron sieve, and/or a 100 micron sieve.

Following particulation, the DBM is treated to remove mineral from the bone as discussed above. While hydrochloric acid is commonly used as a demineralization agent, there are other methods for preparing DBM, which vary widely and include choices regarding the concentration of the demineralization agent; the temperature and duration of the demineralization step; the inclusion or exclusion at various points in the demineralization process of solvents or solvent combinations such as ethanol, methanol, and chloroform:ether; the extent to which the matrix is washed following the demineralization step; and whether the resulting DBM is stored frozen or is lyophilized and stored at room temperature. See, for example, Russell et al., Orthopaedics 22(5):524-53 1, May 1999; incorporated herein by reference.

Any of a variety of DBM preparations may be used with the method disclosed herein. DBM prepared by any method may be employed, including particulate or fiber-based preparations, mixtures of fiber and particulate preparations, fully or partially demineralized preparations, mixtures of fully and partially demineralized preparations, surface demineralized preparations, and combinations of these. See U.S. Pat. No. 6,326,018, Reddi et al., Proc. Natl. Acad. Sci. USA (1972) 69:1601-1605; Lewandrowski et al., Clin. Ortho. Rel. Res., (1995) 317:254-262; Lewandroski et al., J. Biomed. Mater. Res. (1996) 31:365-372; Lewandrowski et al. Calcified Tiss. Int., (1997) 61:294-297; Lewandrowski et al., I Ortho. Res. (1997) 15:748-756, each of which is incorporated herein by reference. Suitable demineralized bone matrix compositions are described in U.S. Pat. No. 5,507,813, hereby incorporated by reference. In some instances, large fragments or even whole bone may be demineralized, and then particulated following demineralization.

b. Neutralizing the Demineralized Bone

Optionally, after demineralization but before treatment with collagenase, the bone may be neutralized. Such neutralization may comprise treating the DBM with phosphate-buffered saline (PBS). For example, in one embodiment, 1 g of DBM is placed in 30 ml of PBS (pH7.5) and agitated for approximately 30 minutes. Neutralization of the DBM before collagenase treatment may provide additional consistency. In some situations, a neutral pH may be used to enhance collagenase action. Thus, neutralizing the bone to a neutral pH may enhance collagenase action.

In some embodiments, a buffer may contain the collagenase. The buffer may be such that it drops the pH of the bone. PBS may then be used to bring the pH of the bone to neutral levels. In other embodiments, the volume of the buffer may be increased to neutralize the pH of the bone.

IV. Collagenase Treatment

The demineralized bone matrix is treated with collagenase. Treatment may comprise partial digestion of the cortical DBM with collagenase, shown at block 12 of FIG. 1. As a general matter, if DBM is treated with collagenase for too long, the DBM will exhibit no osteoinductive activity. In one embodiment, therefore, the DBM is treated with collagenase for approximately one hour. The bone may be treated for any suitable time period, including from about 15 minutes to about 3 hours, from about 30 minutes to about 90 minutes, or for about 60 minutes. Bone from xenogenic sources may be more resistant to digestion by collagenase than bone from human sources. Thus, as a general matter, it may be desirable to treat bone from xenogenic sources for a longer dwell time, for example, for 1, 2, 4, or 6 hours, or for any other suitable period of time. One skilled in the art can readily determine other suitable processing times via routine experimentation.

Collagenase treatment comprises limited digestion with purified bacterial collagenase. Collagenases and their activity on collagens of various types have been extensively studied. A number of collagenase preparations are available from Worthington Biochemical Corporation, Lakewood, N.J. As is known in the art, collagen consists of fibrils composed of laterally aggregated, polarized tropocollagen molecules (MW 300,000). Each tropocollagen unit consists of three helically wound polypeptide a-chains around a single axis. The strands have repetitive glycine residues at every third position and numerous proline and hydroxyproline residues, with the particular amino acid sequence being characteristic of the tissue of origin. Tropocollagen units combine uniformly to create an axially repeating periodicity. Cross linkages continue to develop and collagen becomes progressively more insoluble and resistant to lysis on aging. Gelatin results when soluble tropocollagen is denatured, for example on mild heating, and the polypeptide chains become randomly dispersed. In this state the strands readily may be cleaved by a wide variety of proteases.

Collagenase treatment of cortical human DBM increases its histological score in an in vitro assay without affecting its histological score in an in vivo assay. Generally, it is preferable that the DBM be only partially solubilized by the collagenase.

A variety of different collagenases known in the art can be used. Collagenases are classified in section 3.4.24 under the International Union of Biochemistry and Molecular Biology (NC-IUBMB) enzyme nomenclature recommendations (see, e.g., 3.4.24.3, 3.4.24.7, 3.4.24.19). The collagenase can be of eukaryotic (mammalian) or prokaryotic (bacterial) origin. Bacterial enzymes differ from mammalian collagenases in that they attack many sites along the helix. Collagenase may cleave simultaneously across all three chains or attack a single strand. The collagenase may cleave Type I collagen, e.g., degrade the helical regions in native collagen, such as at the Y-Gly bond in the sequence Pro-Y-Gly-Pro-, where Y is most frequently a neutral amino acid. This cleavage yields products susceptible to further peptidase digestion. Any protease having one or more of these activities associated with collagenase may be used as a collagenase.

It will be appreciated that crude collagenase preparations contain not only several collagenases, but also a sulfhydryl protease, clostripain, a trypsin-like enzyme, and an aminopeptidase. This combination of collagenolytic and proteolytic activities is effective at breaking down intercellular matrices, the essential part of tissue disassociation. Crude collagenase is inhibited by metal chelating agents such as cysteine, EDTA, or o-phenanthroline, but not DFP. It is also inhibited by α2-macroglobulin, a large plasma glycoprotein. Ca²⁺ aids in enzyme activity. Therefore, it may be desirable to avoid collagenase inhibiting agents when treating DBM with collagenase. In addition, although the additional proteases present in some collagenase preparations may aid in breaking down tissue, they also may cause degradation of desired matrix constituents such as growth factors. Therefore, it may be desirable to use a highly purified collagenase that contains low secondary proteolytic activity along with high collagenase activity. For example, a collagenase preparation may contain at least 90%, at least 95%, at least 98%, or at least 99% collagenase by weight. The preparation may be essentially free of bacterial components, particularly bacterial components that could cause inflammatory or immunological reactions in a host, such as endotoxin, lipopolysaccharide, etc. Preparations having a purity greater than 99.5% can be used. It may be desirable to include various protease inhibitors that do not inhibit collagenase but that inhibit various proteases that digest BMP. For example, protease inhibitors that are known to protect BMP activity from degradation include N-ethyl maleimide, benzamidine hydrochloride, iodoacetic acid, PMSF, AEBSF, and E-64. Bestatin may also be used, particularly if the preparation contains aminopeptidase activity. Any of these protease inhibitors (or others) can be included in a carrier, such as a bone matrix composition, or in any composition that is used to treat a carrier.

In alternative embodiments, any suitable compound, for example a chemical compound, for altering the structure of the DBM may be used. For example, enzymes (e.g., pepsin) or chemicals may be used. Pepsin alters the structure of Type I collagen by cleaving the associated telopeptides.

Another suitable protease is bone morphogenetic protein 1 (BMP-1). BMP-1 is a collagenolytic protein that has also been shown to cleave chordin (an inhibitor of BMP-2 and BMP-4). Thus, BMP-1 may be of use to alter the physical structure of the DBM (e.g., by breaking down collagen) and/or to cleave specific inhibitory protein(s), e.g., chordin or noggin. Proteins related to any of the proteases described herein, i.e., proteins or protein fragments having the same cleavage specificity, also can be used. It will be appreciated that variants having substantial sequence identity to naturally occurring proteases can be used. For example, variants at least about 80% identical over at least about 50%, at least about 60%, at least about 70%, at least about 80%, at least about 90%, or about 100% of the length of naturally occurring protease (or any known active fragment thereof that retains cleavage specificity) when aligned for increased identity allowing gaps can be used.

Certain suitable proteases include members of the proprotein convertase (PPC) family of proteases, such as furin and related proteases. Members of this family of cellular enzymes cleave most prohormones and neuropeptide precursors. Numerous other cellular proteins, some viral proteins, and bacterial toxins that are transported by the constitutive secretory pathway are also targeted for maturation by PPCs. Furin and other PPC family members share structural similarities that include a heterogeneous ˜10 kDa amino-terminal proregion, a highly conserved ˜55 kDa subtilisin-like catalytic domain, and carboxyl-terminal domain that is heterogeneous in length and sequence. These enzymes become catalytically active following proregion cleavage within the appropriate cellular compartment. Furin is the major processing enzyme of the secretory pathway and is localized in the trans-golgi network. van den Ouweland, A. M. W. et al. (1990) Nucl. Acid Res. 18, 664; Steiner, D. F. (1998) Curr. Opin. Chem. Biol. 2, 31-39. Substrates of furin include blood clotting factors, serum proteins, and growth factor receptors such as the insulin-like growth factor receptor. Bravo D. A. et al. (1994) J. Biol. Chem. 269, 25830-25873. The minimal cleavage site for farin is Arg-X-X-Arg. However, the enzyme prefers the site Arg-X-(Lys/Arg)-Arg. An additional arginine at the P6 position appears to enhance cleavage. Krysan D. J. et al. (1999) J. Biol. Chem. 274, 23229-23234. Furin is inhibited by EGTA, αl-antitrypsin Portland, Jean, F. et al. (1998) Proc. Natl. Acad. Sci. USA 95, 7293-7298, and polyarginine compounds, Cameron, A. et al. (2000) J. Biol. Chem. 275, 36741-36749. Furin has been shown to proteolytically process both proTGF and proBMP proteins, for example, proTGF-β and proBMP-4, respectively, resulting in the release of the active mature form for each molecule. Dubois et al., American Journal of Pathology (2001) 158(1):305-316; Cui et al., The Embo. Journal (1998) 17(16):47354743; Cui et al., Genes & Development (2001) 15:2797-2802, each incorporated by reference herein. Furin has also been shown to cleave BMP-2, BMP-6, and BMP-7. For example, furin cleaves between amino acids 282 and 283 in mature human BMP-2. Newly synthesized human BMP-2 contains a signal sequence (amino acids 1-23), a propeptide (amino acids 24-282), and an active portion (amino acids 283-396). Furin cleaves mature BMP-2 (amino acids 24-396) between amino acids 282 and 283 to release the propeptide and the active molecule.

Thus, the DBM may be treated with PPCs such as furin and/or other proteases, which process immature TGF-β and/or BMP superfamily propeptides into their active mature forms and/or process active or inactive TGF-β and/or BMP superfamily polypeptides into smaller active fragments that are resistant to degradation or inactivation relative to the longer polypeptide, generating DBM with increased osteoinductivity compared to DBM lacking the protease, resulting in improved bone formation. The higher titers of the mature and/or degradation resistant species in these preparations increase the osteoinductive capacity of the DBM.

V. Assessment of Osteogenic Activity

Osteoinductivity is assessed in tissue culture, e.g., as the ability to induce an osteogenic phenotype in culture cells (primary, secondary, cell lines or explants). The increase in biological activity may be assessed using any of a variety of in vitro methods. Cell culture assays measure the ability of a matrix to cause relatively undifferentiated mesenchymal lineage cells to display one or more features indicative of differentiation along an osteoblastic or chondrocytic lineage. The feature(s) can be expression of a marker characteristic of differentiation along an osteoblastic or chondrocytic lineage, e.g. a marker that is normally expressed by osteoblast precursors, osteoblasts, chondrocytes, or precursors of chondrocytes. One suitable marker is alkaline phosphatase.

One embodiment provides tissue culture assays useful for assessing the osteogenic ability of cortical DBM treated with collagenase. The DBM is exposed to collagenase. Cells, such as a cell culture media, are then exposed to the DBM. Such exposure may comprise adding a dose of DBM to a cell culture media. The exposure can continue for any suitable dwell time, e.g., minutes, hours, days, etc. Further, the cells may be exposed to a second dose of DBM. The assay comprises testing the ability of the cell to (i) express a marker indicative of differentiation along a lineage typical of bone and/or cartilage-forming cells, e.g., an osteoblast, osteocyte, chondroblast, and/or chondrocyte lineage; and/or (ii) display a morphological characteristic indicative of differentiation along a lineage typical of bone and/or cartilage-forming cells, e.g., an osteoblast, osteocyte, chondroblast, and/or chondrocyte lineage; and/or (iii) fail to express a marker characteristic of a lineage other than a lineage typical of bone and/or cartilage-forming cells under conditions in which such expression would otherwise be observed; and/or (iv) fail to display a morphological characteristic indicative of differentiation along a lineage other than a lineage typical of bone and/or cartilage-forming cells. Cell phenotype and/or marker expression can be assessed in the presence or absence of the matrix and can be assessed at any time following exposure of the cells to the matrix. Testing the ability of the cells may be done by lysing the cell culture and assaying the lysate for specific activities, such as alkaline phosphatase activity.

Thus, in accordance with one embodiment, the bone forming potential of DBM may be assessed by exposing the cell culture to the collagenase-treated DBM. The cells may be exposed to the DBM, for example, by adding the DBM to a tissue culture vessel containing the cells, by plating the cells on a matrix surface, etc. The DBM is added to a cell culture, shown at block 14 of FIG. 1.

In one embodiment, the cell culture is exposed to a first dose of collagenase treated DBM, for example, 3 hours after the cells have been seeded in tissue culture dishes. The cell culture is then exposed to a second dose of collagenase treated DBM, for example, 48 hours after the cells have been seed in tissue culture (thus, approximately 45 hours after the first dose). Generally, the second dose may be given approximately when the cells are replaced with fresh media. As will be described more fully below, the cells may then be lysed, for example 96 hours after seeding, and the lysate may be assayed.

Suitable cells for performing the assay include, e.g., mesenchymal stem cells, mesenchymal cells, preosteoblastic cells, etc. As is known in the art, undifferentiated mesenchymal cells are able to differentiate along osteoblastic, chondrocyte, adipocyte, or myocyte pathways to form osteoblasts, chondrocytes, adipocytes, or myocytes. In general, mesenchymal cells suitable for use in the assay can be any cell line that is capable of differentiating along an osteoblast or chondrocyte lineage under appropriate conditions, e.g., when exposed to the appropriate growth factor(s), serum, etc. For example, suitable cells for use in the assay express osteoblast or chondroblast markers when exposed to osteoinductive growth factors. In some embodiments, relatively undifferentiated mesenchymal cells are used. Cell lines (such as clonal cell lines) or primary cells can be used. Primary cells are nonimmortalized cell lines that are recovered directly from an animal and grown for a limited number of passages. The cells may be from any species, e.g., rodent (murine, rat, etc.), primate (monkey or human, etc.), dog, etc. In certain embodiments the cells are selected from the group consisting of W20-17, C2C12, C3H10T1/2, MC3T3-E1, RCJ, 2T3, and ST2 cells. Suitable cell lines are widely available among those of skill in the art. A number of suitable cell lines can be obtained from depositories such as the America Type Culture Collection (ATCC), Manassas, Va., 20108.

After a dwell time (shown at block 16 of FIG. 1) after the DBM is added to the cell line, the cell line is assessed for induction of osteogenic phenotypic markers, genes associated with osteogenesis, or surrogates (such as cartilage), shown at block 18 of FIG. 1. The osteogenic phenotypic marker may be an enzyme such as alkaline phosphatase. Thus, in one embodiment, the DBM is added to a C2C12 culture and, after a dwell time, the C2C12 culture is assessed for induction of alkaline phosphatase. Measuring the presence of alkaline phosphatase, shown at block 20 of FIG. 1, gives an indication of the bone forming potential of the DBM. Suitable markers whose expression can be measured include, but are not limited to, alkaline phosphatase, Osterix, Cbfa-1 (core binding factor 1), dlx-5 (distal-less homeobox 5), MSX2, osteopontin, bone sialoprotein, osteocalcin, osteoblast specific factor 1, RANK ligand, Osteoprotegrin, Collagen Type I, etc. Any suitable measurement method can be used to measure expression of the marker, e.g., assaying an enzymatic reaction, immunological detection of protein, measuring mRNA levels, polymerase chain reaction, etc. The measurement can be qualitative (e.g., whether the marker is or is not detectable), semi-quantitative (e.g., +, ++, +++, with the number of + symbols correlating to the expression level), or quantitative (numerical).

A score for bone forming potential is assigned, shown at block 22 of FIG. 1, based on the induction of osteoblastic markers.

The dwell time between addition of the DBM to the cell culture and assessment for osteogenic phenotypic markers may vary. Any suitable dwell time may be used. The dwell time may vary from 1 day to longer. The dwell time may be between three and 15 days. In one embodiment, the cell culture is assessed after 7 days. Generally, formation of phenotypic markers saturates at approximately 28 days. In some embodiments, the dwell time may be longer than about 28 days, such as where DBM is used as a growth source for growing tissues in vitro (e.g. growing bone or cartilage in cell culture).

A score is assigned based on the presence of phenotypic markers. In some embodiments, the assigned score may be a quantitative score. For example, when measuring the level of alkaline phosphatase induction, the level of AP induced by targeted DBM is compared to the level of AP induced by untreated DBM or tissue culture media express. This may provide a normalized score relative to the amount of AP expressed by untreated DBM.

In alternative embodiments, a score is assigned based on a scale of 1 to 100. A score of 1 is associated with inactive materials, such as DBM extracted with guanidine hydrochloride where the growth factors have been removed. A score of 100 is associated with recombinant BMP at half strength. DBM treated with collagenase will typically score between approximately 50 and 100.

If desired, the tissue culture method can be correlated with an in vivo ectopic bone formation assay, e.g., as described by Zhang et al., “A quantitative assessment of osteoinductivity of human demineralized bone matrix” J Periodontal 68(11):1076-84, November 1997; incorporated herein by reference. Calibration of the in vitro assays against a proven in vivo ectopic bone formation model may be used to confirm that the ability of a compound to induce an apparent “osteogenic” phenotype in tissue culture is correlated with the induction of new bone formation in vivo. Certain BMPs, IGF, TGF-β, and various angiogenic factors are among the osteoinductive factors found to recruit cells from the marrow or perivascular space to the site of injury and then cause the differentiation of these recruited cells down a pathway responsible for bone formation. For example, DBM isolated from either bone or dentin have been found to be osteoinductive materials. Ray et al., “Bone implants” J. Bone Joint Surgery 39A: 1119, 1957; Urist, “Bone: formation by autoinduction” Science 150:893, 1965; each of which is incorporated herein by reference.

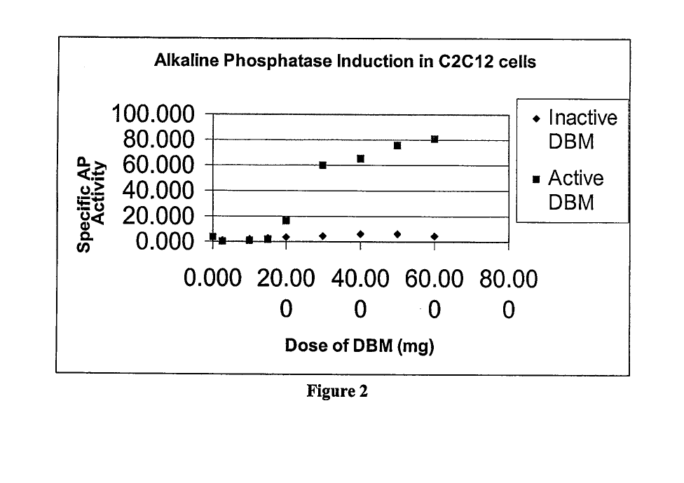

The in vitro assay as taught herein is generally insensitive to noise. More specifically, the in vitro assay does not respond significantly to high doses of inactive DBM, and thus distinguishes between signal (active DBM response) and noise (inactive DBM). For example, as shown in FIG. 2, inactive DBM treated as taught herein induces insignificant alkaline phosphatase activity in C2C12 cells, while active DBM as taught herein induces alkaline phosphatase activity in C2C12 cells. Table 1 below illustrates this characteristic of the in vitro assay.

TABLE 1

| | Inactive DBM | Active DBM | |
|---|---|---|---|
| | AP Activity | AP Activity | Activity |
| 2 doses | (nmoles PNP/min/ | (nmoles PNP/min/ | Ratio |
| (mg/well) | mg protein) | mg protein) | Active/IA |
| 0.000 | 3.885 | 3.885 | 1 |
| 2.500 | 1.250 | 0.419 | 0.34 |
| 10.000 | 2.060 | 0.970 | 0.47 |
| 15.000 | 2.652 | 1.783 | 0.67 |
| 20.000 | 3.257 | 16.558 | 5.08 |
| 30.000 | 4.595 | 60.366 | 13.14 |
| 40.000 | 6.287 | 65.089 | 10.35 |
| 50.000 | 5.953 | 75.756 | 12.73 |
| 60.000 | 3.926 | 80.533 | 20.51 |

### VI. EXAMPLES

Aspects of the present teachings may be further understood in light of the following examples, which should not be construed as limiting the scope of the present teachings in any way.

### Example I

DBM was briefly digested with collagenase. The residual DBM was tested for activity in C2C12 culture.

Methods:

Prepare DBM

1. Prepare an 80 unit/ml collagenase buffer (41.7 μg Worthington CLSPA—clostridium histolyticum collagenase) by adding 200 μl 2000 unit/ml stock to 4.8 ml 50 mM Tris, ph 7.4 containing 5 mM CaCl₂.

2. Add 3 ml Digestion solution to 1 gram human DBM.

3. Incubate in Digestion Buffer for 1 hour at 37° C.

4. At end of 1 hour period, add contents to acetic acid tube. Centrifuge at 2000 rpm and discard supernatant.

5. Wash residual DBM for 60 minutes in 0.1 N acetic acid at 4° C. To wash, add contents of tube to Falcon tube (50 ml) containing 30 ml sterile 0.1 N acetic acid.

6. Aspirate supernatant.

7. Wash residual DBM with 45 ml cold water for 30 minutes.

8. Repeat wash and centrifugation.

9. Wash for 30 minutes in 45 ml cold sterile PBS at 4° C.

10. Aspirate supernatant.

11. Freeze and −80° C.

Prepare C2C12 Cell Culture

1. Prewarm 15 ml High Glucose DMEM, 10% FBS to 37° C., 5% CO₂ for 30 minutes.

2. Remove 1×10⁶ Passage 5 C2C12 cells from liquid nitrogen canister and thaw in 37° C. water.

3. Add cells to 75 cm² flask containing Dulbecco's Modified Eagle's Medium (DMEM) and 5% Fetal Bovine Serum (FBS).

Prepare Aliquots

1. Neutralize 100 mg aliquots of untreated human DBM in a 24 well plate by adding 1 ml aliquots of sterile PBS (4×) and aspirating media after approximately 3 minutes.

2. Rinse with DMEM 10% FBS.

Prepare Treatment Media Containing 10 mM β-glycerophosphate (BGP) and 0.284 mM ascorbate-2-phosphate (Asc).

1. STOCK 1—Prepare a 100×BGP solution by adding 216.04 mg glycerol-2-phosphate salt hydrate to 1 ml 10% FBS in DMEM.

2. STOCK 2—Prepare a 100× (28.4 mM) solution of 2-phospho-L-ascorbic acid trisodium salt by adding 9.146 mg of powder to enough 10% FBS DMEM to achieve a final volume of 1.0 ml.

3. Add 300 μl of STOCK 1 and 300 μl of STOCK 2 to 29.4 ml DMEM, 10% FBS, PSG.

4. Sterile Filter the solution.

Seeding of Cells

1. Remove media from PS C2C12 cells.

2. Rinse 75 cm² flask with 10 ml PBS.

3. Add 2 mls trypsin to flask.

4. Incubate 10 minutes.

5. Transfer cells to 15 ml tube.

6. Spin for 5 minutes at 1000 rpm.

7. Remove media from pellet.

8. Add 1 ml media to cells, gently breaking up pellet by trituration.

9. Transfer cells to 2 ml tube.

10. Triturate up and down to thoroughly break up pellet—approximately 10×.

11. Add 200 μl PBS and 250 μl Trypan Blue to 50 ul cells (1:10 dilution).

12. Add 10 μl to each side of Hemacytometer.

13. Perform cell count.

14. Mix wells and add with the desired number of cells (e.g., 50,000) to each well.

Culturing of the Cells

1. Add Transwell Inserts (Falcon 353097) containing 100 mg DBM, or 100 mg collagenase treated DBM hydrated and neutralized with DMEM/5% FBS to appropriate wells. Alternatively, the treatment groups may be added directly to cell culture wells (without inserts). FBS alone and FBS containing 100 ng/ml recombinant human BMP-2 may serve as negative and positive controls, respectively.

2. Place 0.6 ml of media from plate wall in transwall insert

3. Feed cells at 2 days post seeding by adding fresh DMEM containing 10% FBS, PSG, ascorbate-2-phosphate, β-glycerolphosphate.

4. Feed cells at 4 days post seeding by adding fresh DMEM containing 10% FBS, PSG, ascorbate-2-phosphate, β-glycerolphosphate.

5. During each feeding, replenish BMP-2 by adding 100 ng/ml to each BMP treatment well.

Cell Lysis 6 Days Post-Seeding

1. Wash cells 2× in PBS.

2. Lyse in 1.0 ml TXM Buffer.

3. Store cell lysate in 1.5 ml tubes at −70° C.

4. Thaw for AP analysis 7 days post-seeding.

Results

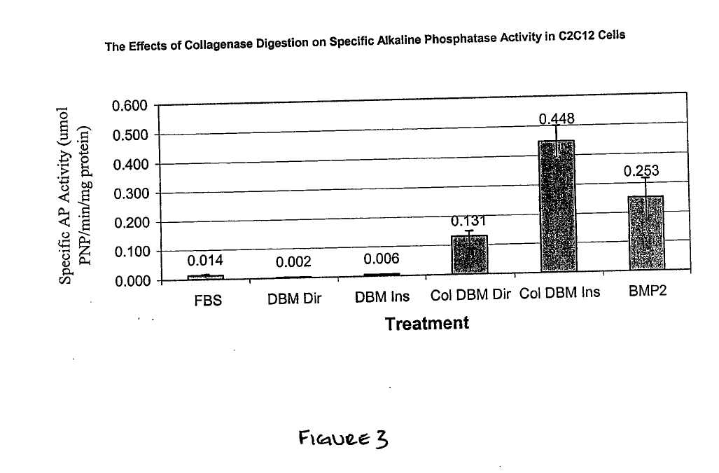

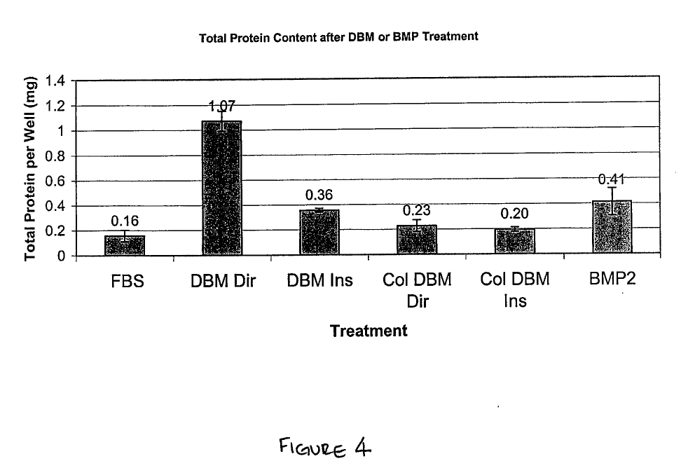

Results of experiment 1 are shown in FIGS. 3 and 4. FIG. 3 illustrates the effects of collagenase digestion on specific alkaline phosphatase activity in C2C12 cells. More specifically, FIG. 3 illustrates the treatment versus the specific AP activity. FIG. 4 illustrates total protein count after DBM or BMP treatment More specifically, FIG. 4 illustrates treatment versus total protein count per well. In FIG. 3 and 4, the following are used:

FBS=Negative control, DMEM containing 5% FBS

DBM Dir=DBM added directly to tissue culture well

DBM Ins=DBM placed in transwell insert

Col DBM Dir=Collagenase treated DBM added directly to tissue culture well

Col DBM Ins=Collagenase treated DBM placed in transwell insert

BMP2=DMEM/5% FBS containing 100 ng/ml recombinant BMP2

### Example 2

### Effects of Collagenase Treatment on DBM Activity and Properties in a Tissue Culture System

Materials and Methods

Preparation of Standard DBM. Methods for preparing demineralized bone matrix have been described previously in the literature. Urist MR, Iwata H, Ceccotti P L, Dorfinan R L, Boyd S D, McDowell R M, Chien C., Bone morphogenesis in implants of insoluble bone gelatin, Proc. Natl. Acad. Sci. USA 1973 December; 70(12):3511-5; Sampath T K, Coughlin J E, Whetstone R M, Banach D, Corbett C, Ridge R J, Ozkaynak E, Oppermann H, Rueger D C, Bovine osteogenic protein is composed of dimers of OP-1 and BMP-2A, two members of the transforming growth factor-beta superfamily, J Biol. Chem. 1990 August 5; 265(22):13198-205. Osteoinductive demineralized human bone matrix was prepared from cortical diaphyseal long bones free from marrow and adhering soft tissues using a method similar to that described in Edwards J T, Diegmann M H, Scarborough N L, Osteoinduction of human demineralized bone: characterization in a rat model, Clin Orthop. 1998 December; 357:219-28. As the osteoinductive growth factors in bone are unstable, the bone was kept in a frozen state prior to the cleaning and the demineralization steps. If the bone is to be cleaned at room temperature, the cleaning may be done quickly (less than 2 hours) to avoid denaturing the osteoinductive factors.

The bones were cut into small cylindrical segments and then powdered using a mill (e.g. Wiley wheat mill, Munson Mill, Fitz Mill). The bone was powered to a size ranging from 106 to 500 μm. The bone powder was defatted in 70% ethanol for 1 hour. Other organic solutions (e.g., a 1:1 solution of chlorofonn/methanol) and/or time periods could be used, e.g., 30 minutes-24 hours. The ethanol solution was poured off and the residue allowed to evaporate away from the bone. No residual organic material was observable following this process.

The defatted bone was then submerged in several volumes (˜15) of 0.6N HCI and allowed to demineralize under agitation. The acid bath was changed at least once to allow demineralization to less than 1% residual calcium. The demineralization was typically carried out at temperatures ranging from 2° C. to 20° C. The process may take from 1 hour to several days, depending on the particle size of the bone, the temperature of demineralization, and the number of times the acid batch is changed; 2 hours was sufficient for the experiments described here. The residual acid was then washed from the bone with several volumes of distilled water and the material was lyophilized.

Collagenase Digestion of DBM. Human DBM 100-500 microns in size was prepared as described above. Some material was inactivated by repeated extraction with 4 M guanidine hydrochloride. Limited digestion with collagenase was carried out as follows: 1 gram of DBM or inactivated DBM was digested for a period of 1 hour at 37° C. in 3 ml of 50 mM Tris-HCI buffer, pH 7.4, containing 5 mM CaCl₂ and 80 units/ml purified bacterial collagenase (Worthington Biochemical, CLSPA collagenase). The residual matrix was then stirred for 1 hour in 45 ml 0.1 N acetic acid at 4° C. After the acid treatment, the matrix was washed twice for 30 minutes with cold water and neutralized by washing for 30 minutes with cold PBS.

As an experimental control, one aliquot of DBM was treated as described, except that collagenase was omitted from the digestion buffer. For all the various bone matrix treatment groups, the equivalent of 100 mg of dry demineralized bone was utilized.

Preparation of Human Bone Matrix Gelatin (BMG). BMG was prepared from osteoinductive DBM by the following method:
1. DBM particles were extracted with 10 volumes of 2 M CaCI₂ at 4° C. for 2 hrs.
2. The material was washed twice for 15 minutes with distilled water.
3. The material was extracted with 10 volumes 0.5 M EDTA, pH 7.4 at 4° C. for 2 hrs.
4. Step 2 was repeated.
5. The material was extracted with 4 volumes of 8 M LiCl at 4° C. for 18 hrs.
6. The material was washed twice with 10 volumes of cold distilled water for 30 minutes.
7. The recovered matrix was placed in sterile water at 55° C. for 1 hr.
8. The matrix was lyophilized.

Tissue Culture and Cell Treatment with DBM. C2C12 mouse myoblastic cells were purchased from ATCC. Passage 6 cells were plated in 24 well plates at a concentration of 30,000 cells per well (depending on experiment). Cells were either grown in Dulbecco's Modification of Eagles Media (Hyclone, SH30243.01) or Minimum Essential Alpha Medium (Gibco 12571-063) supplemented with L-glutamine, Fetal Bovine Serum (Hyclone, SH30071.02) and antibiotics (Penicillin/Streptomycin).

After overnight attachment the cells were exposed to various treatments. During the course of the experiments, 1 ml of culture media was added to each well. Recombinant human BMP-2 (R&D Systems, 355-BM-010) was added to the BMP treatment groups at a concentration of 100 ng/ml. DBM (collagenase treated and untreated, active and inactive) was added to the wells in Falcon 8.0 μm cell culture inserts (Falcon, 353097). Prior to adding the DBM to the tissue culture inserts, it was pre-swollen with tissue culture media. The inserts were placed on top of the cells that adhered to the bottom of the tissue culture well.

Cells were grown for 6 days in a 37° C. incubator where CO₂ concentration was maintained at 5%. The media in each well was replenished at 48 hr intervals. Fresh BMP was added to BMP treatment wells; fresh culture media alone was added to all other wells. The tissue culture inserts containing DBM were temporarily removed for a minimal time period during addition of fresh medium. The DBM in the tissue culture inserts was not removed. No additional DBM was added. The activity of the DBM may decrease over time (e.g., as factors diffuse out of the DBM). Therefore experiments in which the DBM in the tissue culture inserts is replaced during the experiments, e.g., at the time of adding fresh medium, may show more significant effects on alkaline phosphatase expression.

Alkaline Phosphatase Assay. At the end of the treatment period, the cell culture inserts were removed and media was aspirated from all wells. The wells were rinsed three times with phosphate buffered saline and the cells were lysed by adding 1 ml 10 mM Tris-HCl buffer, pH 7.4, containing 1 mM MgCl₂, 20 μM ZnCl₂, and 0.02% Triton x-100 followed by mechanical disruption and followed by three 20 second pulses of sonication on ice (Branson model 1510 sonicator).

The alkaline phosphatase activity of the lysate was then determined by standard techniques. Briefly stated, a known volume of cell lysate (10 μl, 20 μl, or 50 μl depending on particular experiment) was added to 96 well assay plates and the total volume in each well was adjusted to 220 μl by adding 100 mM diethanolamine buffer, pH 10.5, containing 1 mM MgCl₂, and 7.6 mM p-Nitrophenol phosphate (substrate solution). The assay plate was incubated at 37° C. for 30 minutes and the reaction was stopped by addition of 20 μl of 240 mM NaOH. Using a microplate reader, the absorbance of each well was determined at 405 nm. After adjusting for the absorbance of the buffer blank, the alkaline phosphatase activity each sample was determined by comparison to absorbance of known concentrations of p-Nitrophenol standards.

In cases where specific alkaline phosphatase activity is reported, total protein concentration was measured using either the method of Bradford or the Pierce BCA assay.

Evaluating Solubility of DBM. C2C12 cells were initially cultured in the presence of 100 mg standard DBM or collagenase-treated DBM placed in 8.0 μm cell culture inserts. After 6 days of culture, the inserts were removed from the wells containing the cells, and the residual matrix was washed repeatedly with water and then lyophilized. The dry weight of the recovered matrix was measured and reported as percent DBM recovered. The osteoinductivity of the DBM then may be assessed.

Results and Discussion

When prepared properly, e.g., as described herein, demineralized bone matrix has the ability to induce heterotopic bone formation in several animal models including mice, rats, and rabbits. Urist MR., Bone formation by Autoinduction, Science., 1965 Nov. 12; 150(698):893-9. The bone and cartilage forming activity of DBM may be attributed at least in part to the presence of growth factors that diffuse from the matrix and stimulate the differentiation of relatively uncommitted cells along the osteoblastic and chondroblastic lineages. Urist M R, Silverman B F, Buring K, Dubuc F L, Rosenberg J M, The bone induction principle, Clin Orthop., 1967 July-August; 53:243-83. Not all animal species demonstrate similar ability to respond to demineralized bone matrix. In particular, the ability of DBM to induce bone formation in higher order species such as dogs and squirrel monkeys has been questioned. Caplanis N, Lee M B, Zimmerman G J, Selvig K A, Wikesjo U M, Effect of allogenic freeze-dried demineralized bone matrix on guided tissue regeneration in dogs, J Periodontal 1998, August; 69(8):851-6; Aspenberg P, Wang E, Thomgren K G, Bone morphogenetic protein induces bone in the squirrel monkey, but bone matrix does not, Acta Orthop Scand. 1992 December; 63(6):619-22. These species differences could either result from the ability of hosts to respond or from actual differences in the osteoinductive potential of DBM derived from the various species.

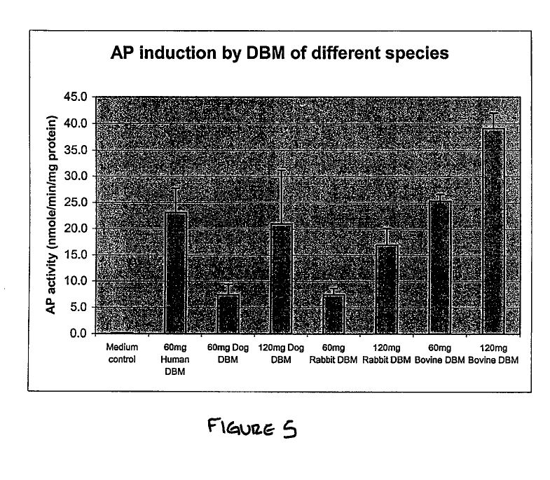

FIG. 5 illustrates alkaline phosphatase induction by DBM of different species. Species shown are human, dog, rabbit, and cow.

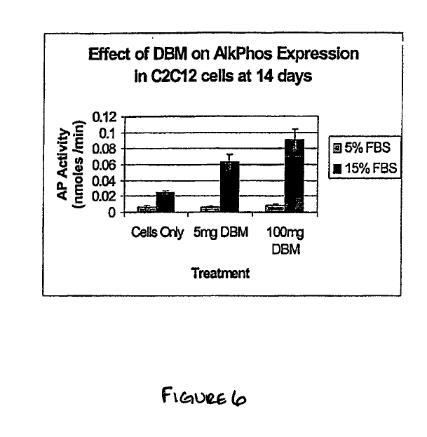

While various preparations of rat DBM have been shown to be effective in inducing cartilage differentiation in primary cultures of neonatal rat muscle, Nogami H, Urist M R, Substrata prepared from bone matrix for chondrogenesis in tissue culture. J Cell Biol. 1974 August; 62(2):510-9, studies have indicated that standard preparations of human DBM, which are of most interest from a therapeutic standpoint, are not particularly potent in vitro. Specifically, human DBM induces only a small increase in the expression of the osteoblast marker alkaline phosphatase in cultures of murine C2C12 or C3H10T1/2 cells. The literature is consistent with these results. For example, in one set of experiments, Han et al. demonstrated only a four fold increase in specific alkaline phosphatase activity of C2C12 cells treated with human DBM over that of cells treated with inactivated DBM. Han B, Tang B, Nimni M E, Quantitative and sensitive in vitro assay for osteoinductive activity of demineralized bone matrix, J Orthop Res. 2003 July; 21(4):648-54. Attempts to replicate the method described in another publication, Peel S A, Hu Z M, Clokie C M, In search of the ideal bone morphogenetic protein delivery system: in vitro studies on demineralized bone matrix, purified, and recombinant bone morphogenetic protein, J Craniofac Surg. 2003 May; 14(3):284-91, yielded inconsistent results. In one experiment, an approximately four fold increase in alkaline phosphatase activity over controls was observed. For example, as shown in FIG. 6, C2C12 cells cultured with DBM using a method corresponding to the work of Peel et al., in the presence of 5% or 15% fetal bovine serum, display only low levels of alkaline phosphatase activity, indicating a lack of significant differentiation along the osteoblast lineage. Those results appear not to be repeatable. Thus it is evident that although rat DBM, rat BMG, and collagenase-treated rat BMG (DBM exposed to LiCl) have chondrogenic potential in vitro, standard human DBM and human bone matrix gelatin (results for BMG not shown) appear to lack such potential. For example, standard human DBM and human bone matrix gelatin lack the ability to induce detectable levels of alkaline phosphatase in clonal cells.

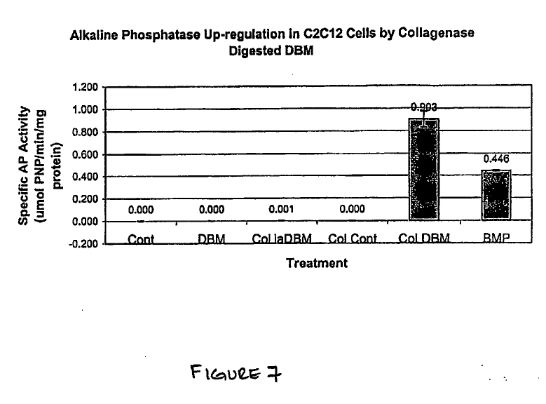

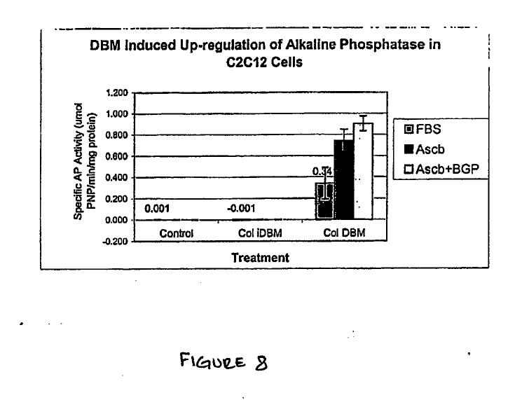

In an effort to increase the activity of human DBM, the material was exposed to collagenase treatment, and the effects of this treatment and others on the osteogenic and/or chondrogenic activity of DBM in a tissue culture system then was assessed. In particular, relatively undifferentiated mesenchymal cells were treated with DBM (treated, untreated, or inactivated), and its effect on alkaline phosphatase activity of the cells was measured. The results indicate that collagenase has a profound effect on the activity of human DBM. In particular, the activity of human DBM in tissue culture can be markedly enhanced if the DBM undergoes limited digestion with purified bacterial collagenase. This increased potency is evidenced by increased expression of alkaline phosphatase activity in cultures of C2C12 cells treated with this modified DBM (FIG. 7). In FIG. 8 it can be seen that the presence of ascorbate 2-phosphate and beta-glycerol phosphate, which may positively influence expression of aspects of the osteoblastic and/or chondroblastic phenotype under certain conditions, enhances but is not essential for visualizing this activity. Standard preparations of human DBM with demonstrated osteoinductive ability in rats fail to induce this phenotype (FIG. 7, DBM group). The data presented graphically in FIGS. 7 and 8 are tabulated below.

TABLE 2
Specific alkaline phosphatase activity of C2C12
cells treated with various preparations of human
DBM, FBS, or BMP-2 (data shown in FIG. 7).

| | Specific AP Activity |
|---|---|
| Treatment | (μmol PNP/min/mg protein) |
| Control (TC media alone) | 0.000 |
| DBM | 0.000 |
| Collagenase Treated Inactive (ia) DBM | 0.001 |
| Digestion Control (DBM incubated | 0.000 |
| with Buffer w/o Collagenase) | |
| Collagenase Treated DBM | 0.903 |
| BMP | 0.446 |

TABLE 3
The effect of Ascorbate 2-phosphate (Ascb) and beta-
glycerol phosphate (BGP) on the in vitro activity of
collagenase treated DBM (data shown in FIG. 8).

| | Specific AP Activity |
|---|---|
| Treatment | (μmol PNP/min/mg protein) |
| TC Media | 0.001 |
| TC Media + Ascb | 0.000 |
| TC Media + Ascb + BGP | 0.000 |
| Collagenase Treated Inactive (ia) DBM | −0.001 |
| Collagenase treated Inactive | 0.001 |
| DBM + Ascb | |
| Collagenase treated Inactive | 0.001 |
| DBM + Ascb + BGP | |
| Collagenase treated DBM | 0.341 |
| Collagenase treated DBM + Ascb | 0.741 |
| Collagenase treated DBM + | 0.903 |
| Ascb + BGP | |

Alkaline phosphatase activity in cells exposed to untreated or inactivated DBM was virtually undetectable. DBM that had been treated with collagenase caused an increase of at least 800-900-fold in alkaline phosphatase activity relative to the effect caused by inactivated collagenase-treated DBM. The fold increase in alkaline phosphatase activity resulting from exposure to collagenase-treated DBM relative to that resulting from (i) exposure to standard DBM or (ii) exposure to collagenase alone or (iii) exposure to tissue culture medium alone was even greater. Since alkaline phosphatase activity in these three control groups of cells was undetectably low, the actual upper bound for the fold increase was probably greater than 900. The increase was approximately 200-450-fold as great as that achieved by exposure of cells to 10% FBS.

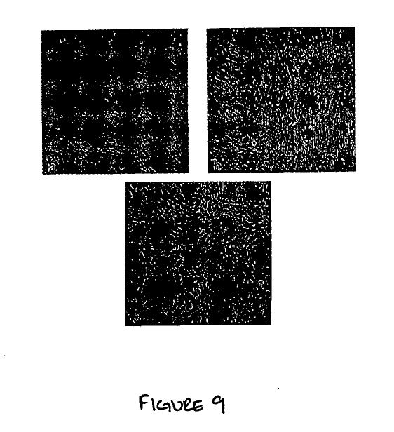

Gross changes in cell phenotype also were observed. Cells treated with collagenase-digested human DBM became round and failed to form myotubes. Changes in cell shape can be seen in FIG. 9. Note the rounded morphology of the cells in FIG. 9c, which were treated with DBM that had been exposed to collagenase, relative to the morphology of the cells in FIG. 9a and 9b, which were treated with either (a) unmodified DBM or (b) collagenase-treated inactivated DBM and exhibit a more elongated appearance.

Alkaline phosphatase activity can be visualized using a variety of substrates, including p-nitrophenol phosphate. Here, it is reported as amount of p-nitrophenol phosphate converted to p-nitrophenol per minute at 37° C. In FIGS. 7 and 8 alkaline phosphatase activity is normalized to total protein content, i.e., the data represents specific alkaline phosphatase activity. Typically, alkaline phosphatase activity is normalized relative to cell number, total protein content, or DNA content. In some cases where standardized cell culture techniques are utilized, alkaline phosphatase activity may be reported per well, per dish, or per volume of cell lysate. Results that are not normalized are considered to be less reliable. Alkaline phosphatase activity may be compared with that in untreated controls, as in these experiments.

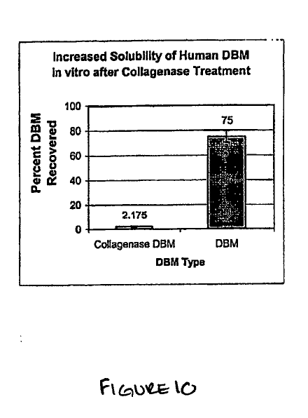

The enhanced activity appears to be correlated with improved solubility of DBM in tissue culture. The solubility of human DBM in tissue culture is markedly enhanced after treatment with collagenase. As seen in FIG. 10, after 6 days of culture collagenase-treated DBM exhibits approximately 34 fold greater solubility than standard DBM preparations. Because the DBM preparations were placed in tissue culture inserts, it is evident that direct cellular contact was not required for solubilization of DBM.

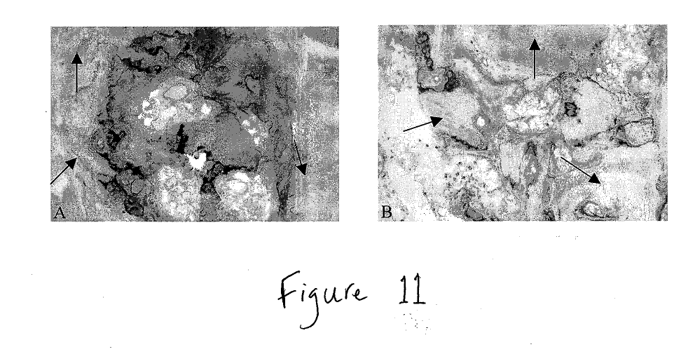

The properties of human bone matrix gelatin prepared according to a method similar to that reported for preparation of rat BMG were evaluated. Nogami and Urist, Transmembrane Bone Matrix Gelatin-Induced Differentiation of Bone, Calcif. Tiss. Res. 1975 19, 153-163; Urist M R, Iwata H, Ceccotti P L, Dorfinan R L, Boyd S D, McDowell R M, Chien C, Bone morphogenesis in implants of insoluble bone gelatin, Proc. Natl. Acad. Sci. USA. 1973 December; 70(12):3511-5. Human BMG did not exhibit the ability to induce significant alkaline phosphatase expression in C2C12 cells. Additionally, empirical observation did not indicate that human BMG had significantly greater solubility in tissue culture media, e.g., as compared with standard human DBM. The effects of implanting human BMG versus human DBM into rat muscle were compared. As shown in FIG. 11, while human BMG is capable of inducing heterotopic bone formation in athymic rats, significant amounts of insoluble residual matrix can be seen 28 days after implantation of either human BMG or human DBM into rat muscle. Without wishing to be bound by any theory, the increased solubility of collagenase-treated human DBM may result in a desirably reduced amount of residual DBM following implantation into a subject.

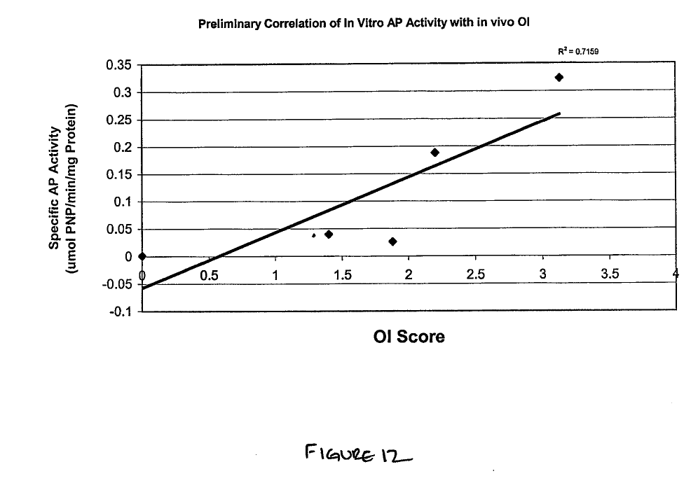

FIG. 12 illustrates a correlation of in vitro alkaline phosphatase activity with in vivo osteoinductivity. As shown, increasing specific alkaline phosphatase activity in vitro correlates to an increasing osteoinductivity score.

Although the method has been described with reference to preferred embodiments, persons skilled in the art will recognize that changes may be made in form and detail without departing from the spirit and scope of the teachings herein.

1. A method for assessing bone forming potential of demineralized bone matrix, comprising:
partially digesting demineralized cortical bone matrix with collagenase;
placing a first dose of the partially digested demineralized cortical bone matrix in a cell culture;
leaving the partially digested demineralized cortical bone matrix in the cell culture for a dwell time;
observing the cell culture for osteoblastic markers.

2. The method of claim 1, wherein partially digesting demineralized cortical bone matrix with collagenase comprises digesting demineralized cortical bone matrix with collagenase for between approximately 15 minutes and approximately 3 hours.

3. The method of claim 1, wherein the demineralized bone matrix is derived from a nonhuman animal source and wherein partially digesting the demineralized cortical bone matrix with collagenase comprises digesting demineralized cortical bone matrix with collagenase for approximately 1 hour, approximately 2 hours, approximately 4 hours, or approximately 6 hours.

4. The method of claim 1, wherein the dwell time is approximately 4 days.

5. The method of claim 1, further comprising lysing the cell culture after the dwell time.

6. The method of claim 1, wherein observing the cell culture for osteoblastic markers comprising observing the cell culture for alkaline phosphatase.

7. The method of claim 1, wherein observing the cell culture for osteoblastic markers comprises measuring the osteoblastic markers in the cell culture.

8. The method of claim 7, wherein measuring the osteoblastic markers is done by polymerase chain reaction.

9. The method of claim 1, wherein the cell culture is a myoblastic cell line culture.

10. The method of claim 5, wherein the myoblastic cell line culture is C2C12.

11. The method of claim 1, further comprising neutralizing the demineralized cortical bone matrix before partially digesting the demineralized cortical bone matrix with collagenase.

12. The method of claim 11, wherein neutralizing the demineralized cortical bone matrix comprises treating the demineralized cortical bone matrix with phosphate-buffered saline.

13. The method of claim 1, wherein the cell culture is seeded and wherein placing a first dose of the partially digested demineralized cortical bone matrix in the cell culture is performed approximately 3 hours after seeding.

14. The method of claim 13, further comprising placing a second dose of the partially digested demineralized cortical bone matrix in the cell culture is performed approximately 48 hours after seeding.

15. The method of claim 14, further comprising adding new media to the cell culture at or near the time of the second dose.

16. The method of claim 14, further comprising lysing the cell culture approximately 96 hours after the seeding.

17. The method of claim 1, wherein, after the dwell time, the cell culture exhibits insignificant osteoblastic markers for inactive demineralized bone matrix.
